(19) United States
(12) Patent Application Publication
(10) Pub. No.: US 2026/0124011 A1
(43) Pub. Date: May 7, 2026

(54) SYSTEMS AND METHODS FOR CONTROL OF A SURGICAL SYSTEM

(71) Applicant: Intuitive Surgical Operations, Inc., Sunnyvale, CA (US)

(72) Inventors: Clifford N. BARGAR, San Francisco, CA (US); J. Scot HART, San Carlos, CA (US); Ashwinram SURESH, San Jose, CA (US); Lawton N. VERNER, Saratoga, CA (US)

(73) Assignee: Intuitive Surgical Operations, Inc., Sunnyvale, CA (US)

(21) Appl. No.: 19/119,536

(22) PCT Filed: Oct. 11, 2023
(86) PCT No.: PCT/US2023/034915

§ 371 Date: Apr. 9, 2025

Related U.S. Application Data
(60) Provisional application No. 63/415,491, filed on Oct. 12, 2022.

(51) Int. Cl. A61B 34/00; A61B 34/30; A61B 90/00

(52) U.S. Cl. CPC A61B 34/74; A61B 34/76; A61B 90/06; A61B 2034/301; A61B 2090/064

(57) ABSTRACT

Systems and methods are provided for control of a surgical system. Accordingly, a first output signal is received from a force sensor unit in response to a first commanded movement of a distal end portion of a medical instrument within a cannula. A force sensor bias value is determined based on a difference between a portion of the first output signal and a baseline output signal for the force sensor. The validity of the force sensor bias value is determined based on a deviation magnitude between the second output signal, which is modified by the force sensor bias value, and the baseline output signal. On a condition that the force sensor bias value is valid, haptic feedback is provided to the user control unit based on a load indication from the force sensor unit as modified by the force sensor bias value.

### CROSS-REFERENCE TO RELATED APPLICATIONS

This application claims priority to and the filing date benefit of U.S. Provisional Ser. No. 63/415,491 , entitled “Systems and Methods for Control of a Surgical System,” filed Oct. 12, 2022, the disclosure of which is incorporated herein by reference in its entirety.

### BACKGROUND

The embodiments described herein relate to surgical systems, and more specifically to teleoperated surgical systems. More particularly, the embodiments described herein relate to systems and methods for determining a force sensor bias value to be applied to a force sensor output in order to control a surgical system that includes a force feedback that may be provided to a system operator.

Known techniques for Minimally Invasive Surgery (MIS) employ instruments to manipulate tissue that can be either manually controlled or controlled via hand-held or mechanically grounded teleoperated medical systems that operate with at least partial computer-assistance (“telesurgical systems”). Many known MIS instruments include a therapeutic or diagnostic end effector (e.g., forceps, a cutting tool, or a cauterizing tool) mounted on an optional wrist mechanism at the distal end of a shaft. During an MIS procedure, the end effector, wrist mechanism, and the distal end of the shaft are typically inserted into a small incision or a natural orifice of a patient via a cannula to position the end effector at a work site within the patient's body. The optional wrist mechanism can be used to change the end effector's position and orientation with reference to the shaft to perform a desired procedure at the work site. In known instruments, motion of the instrument as a whole provides mechanical degrees of freedom (DOFs) for movement of the end effector and the wrist mechanisms generally provide the desired DOFs for movement of the end effector with reference to the shaft of the instrument. For example, for forceps or other grasping tools, known wrist mechanisms are able to change the pitch and yaw of the end effector with reference to the shaft. A wrist may optionally provide a roll DOF for the end effector, or the roll DOF may be implemented by rolling the shaft. An end effector may optionally have additional mechanical DOFs, such as grip or knife blade motion. In some instances, wrist and end effector mechanical DOFs may be combined. For example, U.S. Pat. No. 5,792,135 (filed May 16, 1997) discloses a mechanism in which wrist and end effector grip DOFs are combined.

Force sensing surgical instruments are known and together with associated telesurgical systems may deliver haptic feedback during a MIS procedure to a surgeon performing the procedure. The haptic feedback may increase the immersion, realism, and intuitiveness of the procedure. For effective haptics rendering and accuracy, force sensors may be placed on a medical instrument and as close to the anatomical tissue interaction as possible. One approach is to include a force sensor unit having electrical sensor elements (e.g., strain sensors or strain gauges) at a distal end of a medical instrument shaft to measure strain imparted to the medical instrument. The measured strain can be used to determine the force imparted to the medical instrument and as input upon which the desired haptic feedback may be generated.

Typically, the force sensor unit is calibrated at time of instrument manufacture. This calibration establishes a zero-offset for the force sensing function of the medical instrument—the force sensor unit output that provides an indication that no force is applied to the instrument. During the lifecycle of the medical instrument, however, the zero-offset can shift so that on a condition in which no force is applied to the instrument, the force sensing unit erroneously indicates that a force is applied. For example, the medical instrument is subjected to reprocessing procedures following use that can include exposing the medical instrument, or portions thereof, to relatively high temperatures. This exposure can affect the force sensor unit, resulting in a shift in the zero-offset for the medical instrument. The shift in the zero-offset may, in turn, affect the accuracy of the measured strain used to determine the force imparted to the medical instrument and as input upon which the desired haptic feedback can be generated. Accordingly, it is desirable to determine a correct zero-offset for the medical instrument immediately prior to the medical instrument being used in a surgical procedure in order to provide accurate haptic feedback based on an accurate measure of the strain imparted to the medical instrument.

In view of the aforementioned, the art is continuously seeking new and improved systems and methods for control of a surgical system based on the accurate measurement of the strain imparted to the medical instrument.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

The systems and methods described herein facilitate the accommodation of a deviation of the force sensor unit from a calibration point (e.g., a zero offset) established at time of manufacture. With the accommodation in place, the output of the force sensor unit can be used to generate accurate force feedback and/or can support other functions of a surgical system.

In one aspect, the present disclosure is directed to a surgical system that includes a medical instrument that has a distal end portion. The medical instrument is supported by a manipulator unit that moves the instrument and its distal end portion. A force sensor unit is coupled to the medical instrument to provide indications of forces applied to the instrument at the distal end portion. A user control unit that includes an input device is operably coupled to the medical instrument and to the manipulator unit to allow an operator to move the medical instrument during a medical procedure. A controller is operably coupled to the manipulator unit, the input device, and the force sensor unit to provide a control relationship between these components. The controller includes at least one processor and a haptic feedback module that during a medical procedure provides haptic feedback to the input device based on output from the force sensor unit. The controller is configured to perform a set of operations. The set of operations includes receiving a first output signal from the force sensor unit in response to a first commanded movement of the distal end portion of the medical instrument within a cannula. A force sensor bias value is determined based on a difference between a portion of the first output signal and a baseline output signal for the force sensor. A second commanded movement is initiated and a second output signal is received from the force sensor unit. The second output signal is modified by the force sensor bias value. The validity of the force sensor bias value is determined based on a different magnitude between the second output signal and the baseline output signal. The force sensor bias value is valid on a condition that the deviation magnitude within a predefined tolerance range. Additionally, on a condition that the force sensor bias value is determined to be valid, haptic feedback is provided via the haptic feedback module of the controller to the input device. The haptic feedback is based on a load indication from the force sensor unit as modified by the force sensor bias value.

In some embodiments, on a condition that the force sensor bias value is invalid, an error signal is produced.

In some embodiments, the first commanded movement includes a roll motion of the distal end portion about a longitudinal instrument shaft axis from a first roll limit, through a neutral roll orientation, to a second roll limit, and back to the neutral roll orientation. The distal end portion of the medical instrument is maintained within the cannula throughout the roll motion. Similarly, in some embodiments the first commanded movement includes a linear movement along the longitudinal instrument shaft axis. The distal end portion of the medical instrument is maintained within the cannula throughout the linear movement.

In some embodiments, the manipulator unit includes an instrument carriage on which the medical instrument is mounted, and the instrument carriage includes a set of drive outputs (e.g., discs). Each individual drive output is coupled to a corresponding individual motor of a set of motors. The medical instrument includes a set of instrument drive inputs (e.g., discs). Each individual instrument drive input is configured to engage the corresponding individual drive output. The instrument's drive inputs are configured to receive motion from the manipulator's drive outputs to move the distal end portion. Accordingly, the operations include detecting an installation of the medical instrument on the instrument carriage of the manipulator unit. An engagement process for the medical instrument is automatically initiated in response to detecting the installation. For example, at least one drive output (e.g., a drive output disc) is moved via its corresponding motor until the drive output engages its corresponding instrument drive input (e.g., a drive input disc). Optionally, positive engagement is established by moving the instrument drive input against a mechanical stop. It should be appreciated that in some embodiments, the motors can be included as a component of the instrument.

In some embodiments, the set of drive outputs includes a roll-drive output configured to generate a roll motion of the medical instrument's distal about a longitudinal axis of the instrument's shaft. Accordingly, the operations include maintaining the roll-drive output at a first roll limit while rotating at least one non-roll-drive output to a neutral position, and executing the first commanded movement by generating the roll motion of the instrument's distal end portion through a roll range of motion to a second roll limit.

In some embodiments, the longitudinal position of the distal end portion of the medical instrument within the cannula is a first longitudinal position. The instrument carriage is configured to move the distal end portion of the medical instrument in a proximal direction and in a distal direction within the cannula. Accordingly, the operations include moving the distal end portion of the medical instrument parallel to the instrument shaft's longitudinal axis to a second longitudinal position within the cannula and then returning the distal end portion of the medical instrument to the first longitudinal position within the cannula.

In some embodiments, the operations include determining a difference between a determined magnitude of a force sensor bias value and a defined maximum force sensor bias value. On a condition in which the magnitude of the determined force sensor bias value exceeds the maximum force sensor bias value, an error indication is provided to an operator of the surgical system. In some embodiments, the error indication includes an instruction to remove the medical instrument from the manipulator unit and optionally to reinstall the medical instrument.

In some embodiments, a commanded movement includes establishing the distal end portion of the medical instrument in a first pose, transitioning the distal end portion away from first pose, and returning the distal end portion to first pose. Accordingly, the operations include determining a variability of an output from the force sensor unit between each instance of the distal end portion in the first pose. On a condition in which the variability exceeds a maximum variability value, an error indication is provided to an operator of the surgical system.

In some embodiments, on a condition in which the variability exceeds the maximum variability value, a commanded movement of the distal end portion of the medical instrument within the cannula is repeated to generate a replacement output from the force sensor unit, and the force sensor bias value is determined based at least in part on the replacement output from the force sensor unit.

In some embodiments, a difference of a magnitude of the force sensor bias value relative to a historical force sensor bias value associated with the medical instrument is determined. On a condition in which the difference of these bias values exceeds a deviation threshold, an error indication that indicates a fault with the force sensor unit is provided to an operator of the surgical system.

In some embodiments, a coordinate system for the force sensor unit of the medical instrument is defined to have a first axis, a second axis, and a third axis orthogonal to one another. The force sensor bias value is a first force sensor bias value that is parallel to the first axis. Accordingly, the operations include resolving the output of the force sensor unit in the coordinate system to determine a first axis component, a second axis component, and a third axis component. The operations also include determining a second force sensor bias value parallel to the second axis based on a difference between a portion of the second axis component and a baseline second axis component, and determining a third force sensor bias value parallel to the third axis based on a difference between a portion of the third axis component and a baseline third axis component.

In some embodiments, the controller is configured to execute the set of operations upon receipt of a human command.

In some embodiments, the portion of the first output signal from which the force sensor bias value is determined is the portion of the output signal that is associated with the medical instrument being in a specified sampling pose. In some embodiments, the specified sampling pose includes a roll orientation of the distal end portion of the medical instrument that corresponds to a defined zero orientation.

In some embodiments, determining the force sensor bias value includes identifying a free-space portion of an output from the force sensor that corresponds to a free-space condition of the distal portion of the medical instrument within a cannula. The force sensor bias value corresponds to the difference between an average magnitude of the free-space portion of the output and the baseline output for the force sensor. In some embodiments, the free-space portion of the output corresponds to a portion of the output as a fit-line with a slope less than a defined slope threshold over a specified minimum time interval.

In some embodiments, the operations include determining a confidence score for the free-space portion of the force sensor output. The confidence score is indicative of a correlation between the free-space portion and a condition of the medical instrument in which a commanded movement of the medical instrument is not affected by contact with another object (e.g., the cannula). A command action is implemented based at least in part on the confidence score.

In some embodiments, on a condition in which the confidence score is less than a confidence score threshold, implementing the command action includes repeating the commanded movement of the distal end portion of the medical instrument within the cannula to generate a replacement first output signal. A replacement free-space portion of the first output signal is identified, and the force sensor bias value is determined based at least in part on the replacement free-space portion.

In some embodiments, on a condition in which the confidence score is less than a confidence score threshold, implementing the command action includes providing an error indication to the input device.

In some embodiments, implementing the command action includes applying a gain value to the haptic feedback provided to the input device. The gain value is determined based at least in part on the confidence score. A higher gain value is associated with a higher confidence score, and a lower gain value is associated with a lower confidence score.

In some embodiments, on a condition in which the confidence score is less than a confidence score threshold, implementing the command action includes generating a maintenance alert indicative of a failed or failing force sensor unit.

With regards to the validity of the force sensor bias value, in some embodiments, on a condition in which the difference between the second output signal and the baseline output signal falls outside the tolerance range, an error signal is generated, and a command action is implemented based at least in part on the error signal. In some embodiments, implementing the command action includes delivering an instruction to remove the medical instrument from the manipulator unit and reinstall the medical instrument. In some embodiments, implementing the command action includes delivering an instruction to an operator of the surgical system to remove the medical instrument from service due to a fault condition with the force sensor unit.

In some embodiments, implementing the command action includes repeating the first commanded movement of the distal end portion of the medical instrument within the cannula to generate a replacement first output signal. A replacement force sensor bias value is determined based on a difference between a portion of the replacement first output signal and a baseline output signal for the force sensor, and haptic feedback is provided to the input device based on the load indication from the force sensor unit as modified by the replacement force sensor bias value.

These and other features, aspects and advantages of the present invention will become better understood with reference to the following description and drawings.

### BRIEF DESCRIPTION OF THE DRAWINGS

FIG. 1 is a plan view of a minimally invasive teleoperated medical system according to an embodiment being used to perform a medical procedure such as surgery.

FIG. 2 is a perspective view of a user control console of the minimally invasive teleoperated surgery system shown in FIG. 1.

FIG. 3 is a perspective view of an optional auxiliary unit of the minimally invasive teleoperated surgery system shown in FIG. 1.

FIG. 4 is a front view of a manipulator unit, including a plurality of instruments, of the minimally invasive teleoperated surgery system shown in FIG. 1.

FIG. 5 is an illustration of a portion of the teleoperated system of FIG. 1, illustrating an instrument carriage of the manipulator unit, according to an embodiment.

FIG. 6 is a perspective view of a medical instrument according to an embodiment.

FIG. 7 is a side view of a portion of the medical device of FIG. 6 with an outer shaft removed.

FIG. 8 is a perspective view of a cannula of the minimally invasive teleoperated surgery system shown in FIG. 1.

FIG. 9 is a cross-sectional side view of a portion of the cannula of FIG. 8 with a distal end portion of the medical instrument of FIG. 6 positioned therein in a no-load condition.

FIG. 10 is a cross-sectional side view of a portion of the cannula of FIG. 8 with a distal end portion of the medical instrument of FIG. 6 positioned therein in contact with an obstruction.

FIG. 11 is a flow chart of a set of operations or control of a surgical system.

FIG. 12 is a graph showing an output of a force sensor unit in response to a commanded movement of the distal end portion of the medical instrument of FIG. 6.

FIG. 13 is a schematic illustration of a controller for use with a minimally invasive teleoperated surgery system according to an embodiment.

FIG. 14 is a flow chart of a method of control for a surgical system according to an embodiment.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

The embodiments described herein can advantageously be used in a wide variety of grasping, cutting, and manipulating operations associated with minimally invasive surgery. The medical instruments or devices of the present application enable motion in three or more degrees of freedom (DOFs). For example, in some embodiments, an end effector of the medical instrument can move with reference to the main body of the instrument in three mechanical DOFs, e.g., pitch, yaw, and roll (shaft roll). There may also be one or more mechanical DOFs in the end effector itself, e.g., two jaws, each rotating with reference to a clevis (2 DOFs) and a distal clevis that may rotate with reference to a proximal clevis (one DOF). Thus, in some embodiments, the medical instruments or devices of the present application may enable motion in six DOFs. The embodiments described herein further may be used to deliver haptic feedback to a system operator based on a load indication from the force sensor unit as modified by the force sensor bias value.

Generally, the present disclosure is directed to systems and methods for controlling a surgical system (system) such as a minimally invasive teleoperated surgery system. In particular, the present disclosure includes a system and methods that may facilitate the accurate sensing (e.g., measuring) of loads affecting a medical instrument and the delivery of haptic feedback based on the sensed loads. Accordingly, the systems and methods described herein facilitate the accommodation of a deviation of the force sensor unit from a calibration point (e.g., a zero-offset) established at time of manufacture.

As described herein, the medical instrument is coupled to the manipulator unit of the surgical system and a distal end portion of the medical instrument is positioned within a cannula. While the distal end portion of the medical instrument is within the cannula, the distal end portion executes a first commanded movement. A controller of the system receives a first output signal from a force sensor unit of the medical instrument in response to the first commanded movement. Insofar as the distal end portion is within the cannula, the medical instrument is in a no-load condition while executing the first commanded movement. In a nominal (e.g., designed) no-load condition, the distal end portion is affected by the force of gravity but does not generate an applied or reactive force by contact with another object. In other words, under the no-load condition, the initial zero-offset (e.g., bias) will result in a force sensor output indicating, with only negligible deviations, a load magnitude of “zero” in the absence of drift or other deviation of the zero-offset.

Upon receipt of the first output signal, the controller determines a difference between a portion of the first output signal and a baseline output signal for the force sensor. This difference can correspond to a force sensor bias value (e.g., a corrective value) required to compensate for the deviation (e.g., drift) of the force sensor unit from the initial calibration point. In other words, the difference can be utilized to establish a new zero-offset (e.g., recalibrate the force sensor unit) for the present installation of the medical instrument. Accordingly, the force sensor bias value can be re-calculated each time the medical instrument is coupled to the manipulator unit to ensure an accurate representation of the loads applied to or by the medical instrument.

In order to determine the validity of the force sensor bias value, the controller implements a second commanded movement of the distal end portion within this cannula. Like the first commanded movement, the second commanded movement also corresponds to a no-load condition. A second output signal is received from the force sensor unit in response to the second commanded movement. However, the second output signal is modified by the force sensor bias value. The controller then determines the validity of the force sensor bias value based on a deviation magnitude between the second output signal and the baseline output signal. If the deviation magnitude is within a predefined tolerance range, then the force sensor bias value is valid. For example, if the second output signal indicates a load magnitude of “zero” (with only negligible deviations therefrom) when modified by the force sensor bias value, then the force sensor bias value is valid. If the force sensor bias value is valid, then the force sensor bias value can be applied to load indications from the force sensor unit during operations of the system. When modified by the force sensor bias value, the load indications from the force sensor unit can be utilized to provide haptic feedback to an input device of the system. However, if the force sensor bias value is invalid, the controller generates an error signal, and an operation of the system is modified. For example, in response to the error signal, the medical instrument can be removed and re-coupled to the manipulator unit, the cannula can be inspected for obstructions, the medical instrument can be replaced, the magnitude of the haptic feedback can be limited, and/or other suitable modifications can be implemented.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used in this specification and the appended claims, the word “distal” refers to direction towards a work site, and the word “proximal” refers to a direction away from the work site. Thus, for example, the end of a tool that is closest to the target tissue would be the distal end of the tool, and the end opposite the distal end (i.e., the end manipulated by the user or coupled to the actuation shaft) would be the proximal end of the tool.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like-may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes includes various spatial device positions and orientations. The combination of a body's position and orientation define the body's pose (e.g., a kinematic pose).

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, instrument, and variants thereof, can be interchangeably used.

Inventive aspects are described with reference to a teleoperated surgical system. An example architecture of such a teleoperated surgical system is the da Vinci® surgical system commercialized by Intuitive Surgical, Inc., Sunnyvale, California. Knowledgeable persons will understand, however, that inventive aspects disclosed herein may be embodied and implemented in various ways, including computer-assisted, non-computer-assisted, and hybrid combinations of manual and computer-assisted embodiments and implementations. Implementations are merely presented as examples, and they are not to be considered as limiting the scope of the inventive aspects disclosed herein. As applicable, inventive aspects may be embodied and implemented in both relatively smaller, hand-held, hand-operated devices and relatively larger systems that have additional mechanical support.

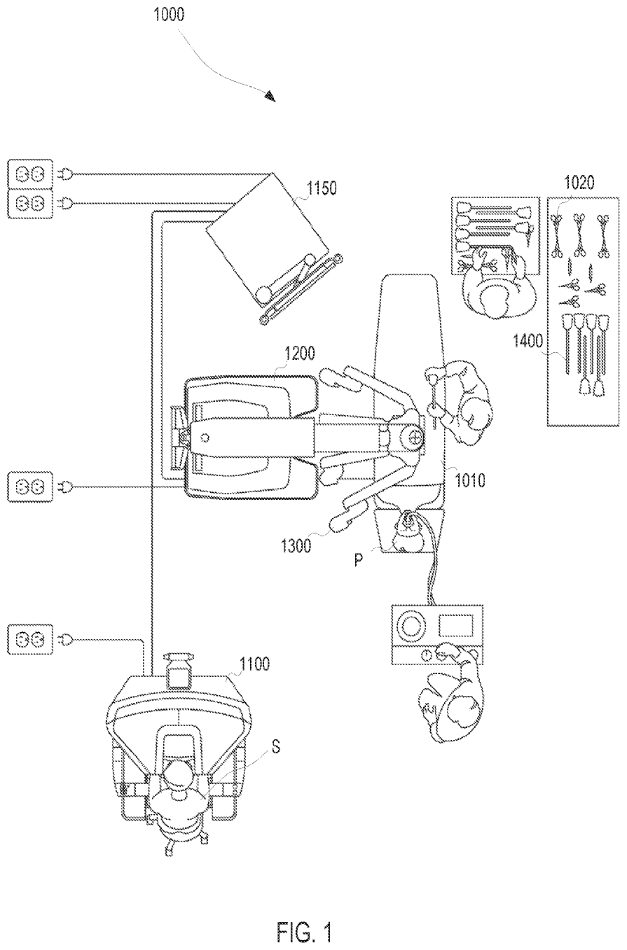

FIG. 1 is a plan view illustration of a teleoperated surgical system (“system”) 1000 that operates with at least partial computer assistance (a “telesurgical system”). Both telesurgical system 1000 and its components are considered medical devices. Telesurgical system 1000 is a Minimally Invasive Robotic Surgical (MIRS) system used for performing a minimally invasive diagnostic or surgical procedure on a Patient P who is lying on an Operating table 1010. The system can have any number of components, such as a user control unit 1100 for use by an operator of the system, such as a surgeon or other skilled clinician S, during the procedure. The MIRS system 1000 can further include a manipulator unit 1200 (popularly referred to as a surgical robot) and an optional auxiliary equipment unit 1150. The manipulator unit 1200 can include an arm assembly 1300 and a surgical instrument tool assembly removably coupled to the arm assembly. The manipulator unit 1200 can manipulate at least one removably coupled medical instrument (instrument) 1400 through a minimally invasive incision in the body or natural orifice of the patient P while the surgeon S views the surgical site and controls movement of the instrument 1400 through control unit 1100. An image of the surgical site is obtained by an endoscope (not shown), such as a stereoscopic endoscope, which can be manipulated by the manipulator unit 1200 to orient the endoscope. The auxiliary equipment unit 1150 can be used to process the images of the surgical site for subsequent display to the Surgeon S through the user control unit 1100. The number of instruments 1400 used at one time will generally depend on the diagnostic or surgical procedure and the space constraints within the operating room, among other factors. If it is necessary to change one or more of the instruments 1400 being used during a procedure, an assistant removes the instrument 1400 from the manipulator unit 1200 and replaces it with another instrument 1400 from a tray 1020 in the operating room. Although shown as being used with the instruments 1400, any of the instruments described herein can be used with the system 1000.

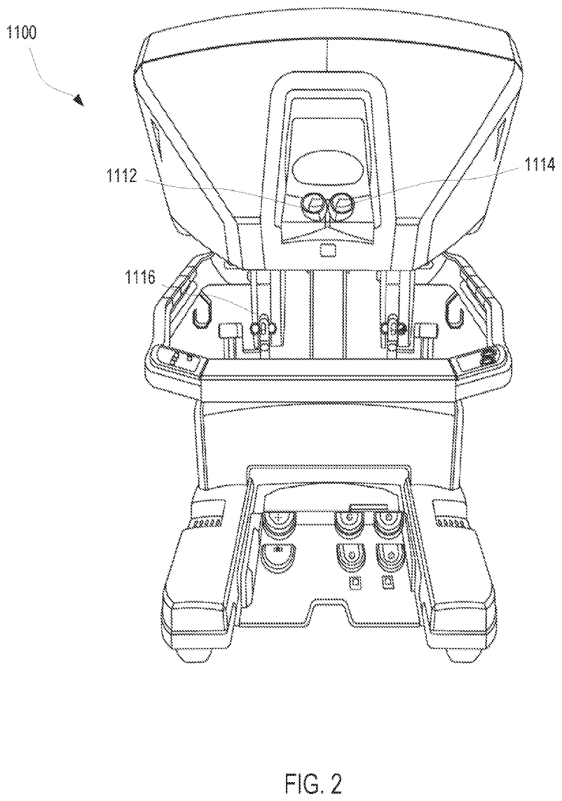

FIG. 2 is a perspective view of the control unit 1100. The user control unit 1100 includes a left eye display 1112 and a right eye display 1114 for presenting the surgeon S with a coordinated stereoscopic view of the surgical site that enables depth perception. The user control unit 1100 further includes one or more input control devices 1116 (input device), which in turn cause the manipulator unit 1200 (shown in FIG. 1) to manipulate one or more tools. The input devices 1116 provide at least the same degrees of freedom as instruments 1400 with which they are associated to provide the surgeon S with telepresence, or the perception that the input devices 1116 are integral with (or are directly connected to) the instruments 1400. In this manner, the user control unit 1100 provides the surgeon S with a strong sense of directly controlling the instruments 1400. To this end, position, force, strain, or tactile feedback sensors (not shown) or any combination of such sensations, from the instruments 1400 back to the surgeon's hand or hands through the one or more input devices 1116.

The user control unit 1100 is shown in FIG. 1 as being in the same room as the patient so that the surgeon S can directly monitor the procedure, be physically present if necessary, and speak to an assistant directly rather than over the telephone or other communication medium. In other embodiments, however, the user control unit 1100 and the surgeon S can be in a different room, a completely different building, or other location remote from the patient, allowing for remote surgical procedures.

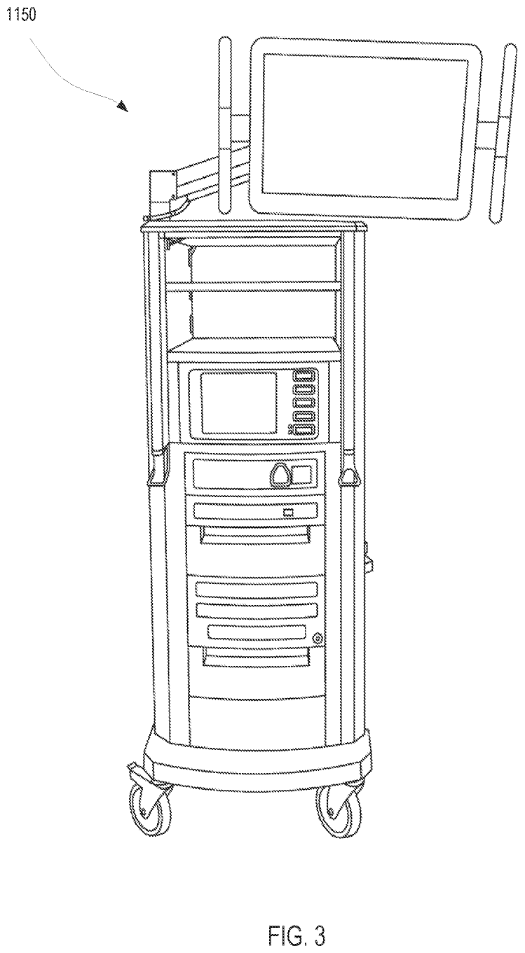

FIG. 3 is a perspective view of the auxiliary equipment unit 1150. The auxiliary equipment unit 1150 can be coupled with the endoscope (not shown) and can include one or more processors to process captured images for subsequent display, such as via the user control unit 1100, or on another suitable display located locally (e.g., on the unit 1150 itself as shown, on a wall-mounted display) and/or remotely. For example, where a stereoscopic endoscope is used, the auxiliary equipment unit 1150 can process the captured images to present the surgeon S with coordinated stereo images of the surgical site via the left eye display 1112 and the right eye display 1114. Such coordination can include alignment between the opposing images and can include adjusting the stereo working distance of the stereoscopic endoscope. As another example, image processing can include the use of previously determined camera calibration parameters to compensate for imaging errors of the image capture device, such as optical aberrations.

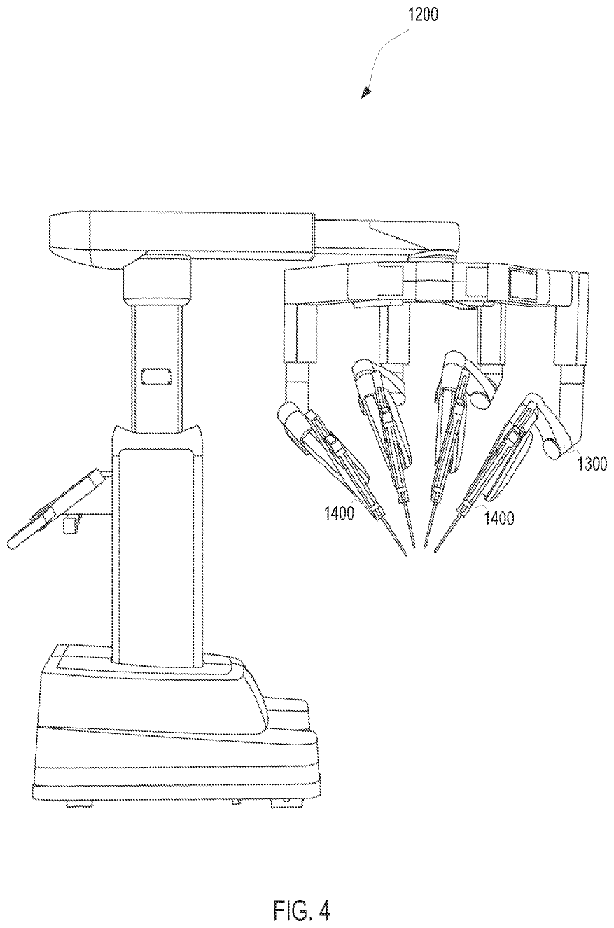

FIG. 4 shows a front perspective view of the manipulator unit 1200. The manipulator unit 1200 includes the components (e.g., arms, linkages, motors, sensors, and the like) to provide for the manipulation of the instruments 1400 and an imaging device (not shown), such as a stereoscopic endoscope, used for the capture of images of the site of the procedure. Specifically, the instruments 1400 and the imaging device can be manipulated by teleoperated mechanisms having one or more mechanical joints. Moreover, the instruments 1400 and the imaging device are positioned and manipulated through incisions or natural orifices in the patient P in a manner such that a center of motion remote from the manipulator and typically located at a position along the instrument shaft is maintained at the incision or orifice by either kinematic mechanical or software constraints. In this manner, the incision size can be minimized.

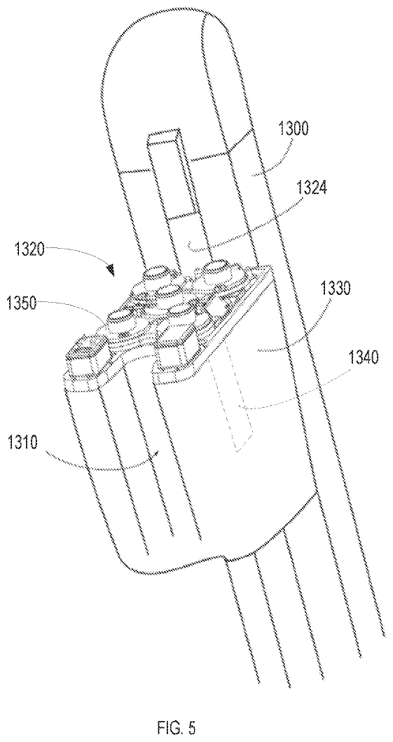

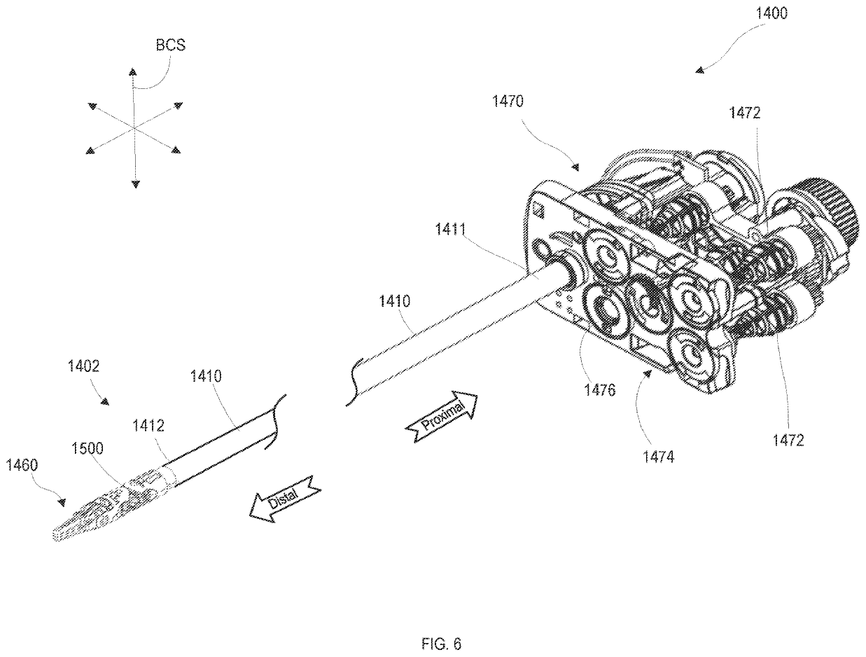

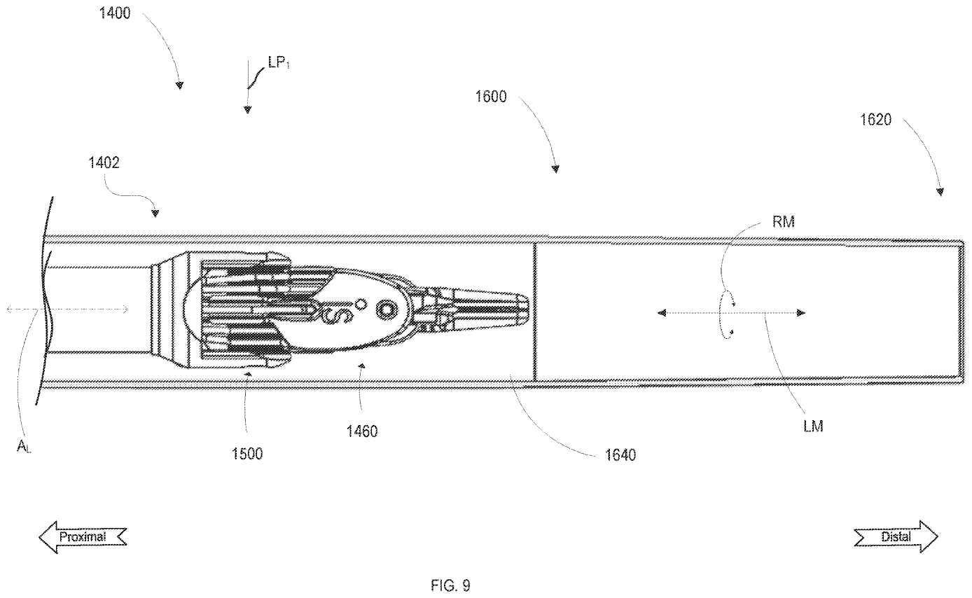

FIG. 5 is a perspective view of a portion of an arm assembly 1300 and an instrument carriage 1330 to which an instrument 1400 can be removably coupled. The instrument carriage 1330 includes teleoperated actuators (e.g., motors 1340 with coupled drive discs 1320) to provide controller motions to the instrument 1400, which translates into a variety of movements of a tool or tools at a distal end portion 1402 (FIG. 6) of the instrument 1400. The arm assembly 1300 includes a connecting portion 1324 in which the instrument carriage 1330 can be coupled. The instrument carriage 1330 may be translatable relative to the arm assembly 1300, for example, along an insertion axis extending between a proximal end and a distal end of the arm assembly 1300 for insertion and removal of the instrument into a patient. The translation of the instrument carriage 1330 can develop a corresponding linear motion LM (see FIG. 9), relative to a longitudinal axis A_{L} (e.g., in a distal or proximal direction) of a distal end portion 1402 (FIG. 6) of the instrument 1400. In addition, the arm assembly 1300 can provide for additional degrees of freedom to orient and position the instrument carriage 1330 and instrument 1400 at a desired location. When an instrument 1400 is coupled to the instrument carriage 1330, input provided by a surgeon S to the user control unit 1100 (a “master” command) is translated into a corresponding action by the instrument 1400(a “slave” response) via drive discs 1320 of the instrument carriage 1330 that are operatively coupled instrument discs (FIG. 6) on the instrument 1400.

The instrument carriage 1330 includes a carriage interface that includes drive discs 1320 that are configured to be operatively coupled with instrument discs 1474 at a drive member interface. In embodiments utilizing a sterile adapter or other similar structure, the drive discs 1320 may be matingly coupled to couplers of the instrument sterile adapter. The instrument carriage 1330 also includes an indentation or cutout region 1310 in which the instrument shaft (shaft) 1410 (FIG. 6) of the instrument 1400 can extend when the instrument 1400 is supported by the manipulator unit 1200. In some embodiments, the drive discs 1320 of the carriage 1330 may be directly coupled to inputs of the instrument discs 1474 of the instrument 1400 without an intermediary sterile adapter.

In some embodiments, the instrument carriage 1330 can include a roll-drive disc 1350. The roll-drive disc 1350 is configured to be operatively coupled to a roll-drive instrument discs 1476 to generate a roll motion RM (FIG. 9) of the distal end portion 1402 of the instrument 1400 about a longitudinal (e.g., shaft) axis A_{L} of the instrument 1400. The roll motion RM has a roll range of motion defined between a first roll limit and a second roll limit. For example, the roll range of motion can include up to 360 degrees (e.g., 350 degrees) of roll in a clockwise direction from a neutral roll orientation (e.g., a zero-degree position) to the first roll limit and up to 360 degrees (e.g., 350 degrees) of roll in a counterclockwise direction from the neutral roll orientation to a second roll limit. Accordingly, the roll range of motion can include 720 degrees (e.g., 700 degrees) of roll from the first roll limit, through the neutral roll orientation to the second roll limit.

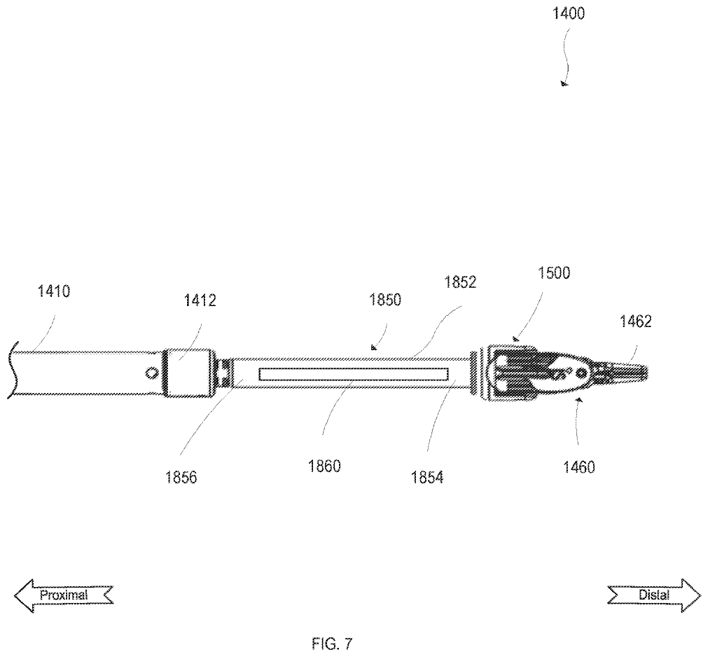

Referring now to FIGS. 6 and 7, a perspective view of the instrument 1400 is depicted in FIG. 6, and a side view of a portion of the instrument 1400 with an outer shaft portion removed is depicted in FIG. 7. In some embodiments, the instrument 1400 or any of the components therein are optionally parts of a surgical system that performs surgical procedures, and which can include a manipulator unit, a series of kinematic linkages, a set of cannulas, or the like. The instrument 1400 (and any of the instruments described herein) can be used in any suitable surgical system, such as the MIRS system 1000 shown and described above. As shown in FIG. 6, the instrument 1400 defines includes a proximal mechanical structure 1470, a shaft 1410, a distal end portion 1402, and a set of cables (not shown). The cables function as tension elements that couple the proximal mechanical structure 1470 to the distal end portion 1402. In some embodiments, the distal end portion 1402 includes a distal wrist assembly 1500 and a distal end effector 1460. The instrument 1400 is configured such that movement of one or more of the cables produces movement of the end effector 1460 (e.g., pitch, yaw, or grip) about axes of a beam coordinate system BCS.

Moreover, although the proximal mechanical structure 1470 is shown as including capstans 1472, in other embodiments, a mechanical structure can include one or more linear actuators that produce translation (linear motion) of a portion of the cables. Such proximal mechanical structures can include, for example, a gimbal, a lever, or any other suitable mechanism to directly pull (or release) an end portion of any of the cables. For example, in some embodiments, the proximal mechanical structure 1470 can include any of the proximal mechanical structures or components described in U.S. Patent Application Pub. No. US 2015/0047454 A1 (filed Aug. 15, 2014), entitled “Lever Actuated Gimbal Plate,” or U.S. Pat. No. 6,817,974 B2 (filed Jun. 28, 2001), entitled “Surgical Tool Having Positively Positionable Tendon-Actuated Multi-Disc Wrist Joint,” each of which is incorporated herein by reference in its entirety.

The shaft 1410 can be any suitable elongated shaft that is coupled to the wrist assembly 1500 and to the proximal mechanical structure 1470. Specifically, the shaft 1410 includes a proximal end 1411 that is coupled to the proximal mechanical structure 1470, and a distal end portion 1412 that is coupled to the wrist assembly 1500 (e.g., a proximal link of the wrist assembly 1500). The shaft 1410 defines a passageway or series of passageways through which the cables and other components (e.g., electrical wires, ground wires, or the like) can be routed from the proximal mechanical structure 1470 to the wrist assembly 1500. In some embodiments, the shaft 1410 can be formed, at least in part with, for example, an electrically conductive material such as stainless steel. In such embodiments, the shaft may include any of an inner insulative cover or an outer insulative cover. Thus, the shaft 1410 can be a shaft assembly that includes multiple different components. For example, the shaft 1410 can include (or be coupled to) a spacer that provides the desired fluid seals, electrical isolation features, and any other desired components for coupling the wrist assembly 1500 to the shaft 1410. Similarly stated, although the wrist assembly 1500 (and other wrist assemblies or links described herein) are described as being coupled to the shaft 1410, it is understood that any of the wrist assemblies or links described herein can be coupled to the shaft via any suitable intermediate structure, such as a spacer and a cable guide, or the like.

As depicted in FIG. 7, the instrument 1400 (e.g., the surgical or medical instrument) includes a force sensor unit 1850 including a beam 1852, with one or more strain sensors 1860. The strain sensor 1860 can include a set of strain gauges (e.g., tension strain gauge resistor(s) or compression strain gauge resistor(s)) arranged as at least one bridge circuit (e.g., Wheatstone bridges) mounted on a surface along the beam 1852. In some embodiments, the end effector 1460 can be coupled at a distal end portion 1854 of the beam 1852 (e.g. at a distal end portion 1402 of the surgical instrument 1400) via the wrist assembly 1500. The shaft 1410 includes a distal end portion 1412 that is coupled to a proximal end portion 1856 of the beam 1852. In some embodiments, the distal end portion 1412 of the shaft 1410 is coupled to the proximal end portion 1856 of the beam 1852 via another coupling component (such as an anchor or coupler, not shown). In some embodiments, the force sensor unit 1850 can include any of the structures or components described in U.S. Patent Application Pub. No. US 2020/0278265 A1 (filed May. 13, 2020), entitled “Split Bridge Circuit Force Sensor,” which is incorporated herein by reference in its entirety.

In some embodiments, the end effector 1460 can include at least one tool member 1462 having a contact portion configured to engage or manipulate a target tissue during a surgical procedure. For example, in some embodiments, the contact portion can include an engagement surface that functions as a gripper, cutter, tissue manipulator, or the like. In other embodiments, the contact portion can be an energized tool member that is used for cauterization or electrosurgical procedures. The end effector 1460 may be operatively coupled to the proximal mechanical structure 1470 such that the tool member 1462 rotates relative to shaft 1410. In this manner, the contact portion of the tool member 1462 can be actuated to engage or manipulate a target tissue during a surgical procedure. The tool member 1462 (or any of the tool members described herein) can be any suitable medical tool member. Moreover, although only one tool member 1462 is identified, as shown, the instrument 1400 can include two tool members that cooperatively perform gripping or shearing functions. In other embodiments, an end effector can include more than two tool members.

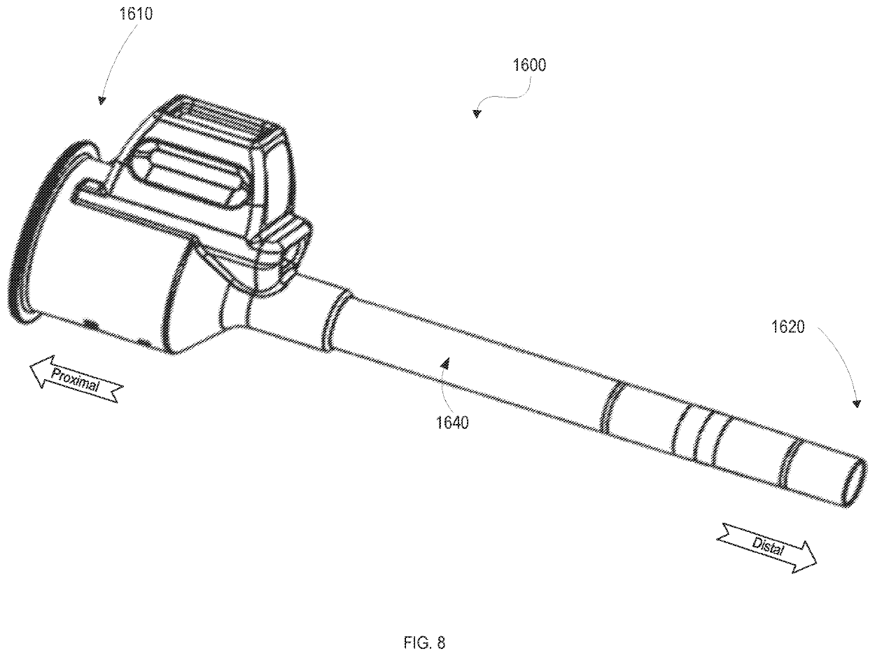

FIG. 8 depicts a cross-sectional view of a cannula 1600 for use with the system 1000. As depicted, the cannula 1600 can be configured to circumscribe at least a portion of the instrument 1400 to facilitate access of the surgical site by the end effector 1460. Accordingly, the cannula 1600 can have a proximal end 1610 and a distal end 1620. A central channel 1640 extends between the proximal and distal ends 1610, 1620. As such, the cannula 1600 forms a channel or passage through which the instrument 1400 can be inserted to access the surgical site. As depicted, the cannula 1600 can be a straight cannula. However, in additional embodiments, the cannula 1600 can, for example, be a curved cannula having a combination of linear and nonlinear sections, a cannula with multiple non-parallel linear sections, a cannula with multiple curve sections having different characters, and/or a cannula with other combinations of linear and nonlinear sections. In some embodiments, the distal end 1620 of the cannula 1600 is inserted through an incision and into a body cavity of the patient. The proximal end 1610 of the cannula 1600 is maintained external to a body wall of the patient and is coupled to the arm assembly 1300 of the system 1000.

FIG. 9 is a cross-sectional side view of a portion of the cannula 1600 with a distal end portion 1402 of the instrument 1400 positioned therein in a no-load condition. As depicted, the distal end portion 1402 is positioned within the central channel 1640 of the cannula 1600 at a first longitudinal position LP₁. When positioned within the cannula, the distal end portion 1402 is separated from the body of the patient. Accordingly, the distal end portion 1402 does not generate an applied or reactive force by contact with a body of the patient and the instrument 1400 is in a nominal no-load condition. Further, in the no-load condition, the distal end portion 1402 is separated from the central channel 1640 and from any other obstruction (e.g., debris such as biologic tissue or a surgical material) within the central channel 1640. Accordingly, in the no-load condition, a movement of the distal end portion 1402 within the central channel 1640 is not affected by contact with the body of the patient, the cannula 1600, nor any obstruction within the central channel 1640. Thus, any output from the force sensor unit 1850 is not related to any external force (except gravity) exerted on the distal end portion 1402. Therefore, in the no-load condition, the movement of the distal end portion 1402 within the central channel can be utilized to confirm or reestablish a zero-offset of the force sensor unit 1850 or the instant installation of the instrument 1400 on the arm assembly 1300. The confirmation or reestablishment of the zero-offset for each installation of the instrument 1400 can facilitate the accurate measurement of loads affecting the instrument 1400 and the provision of haptic feedback to the operator based on the measurements.

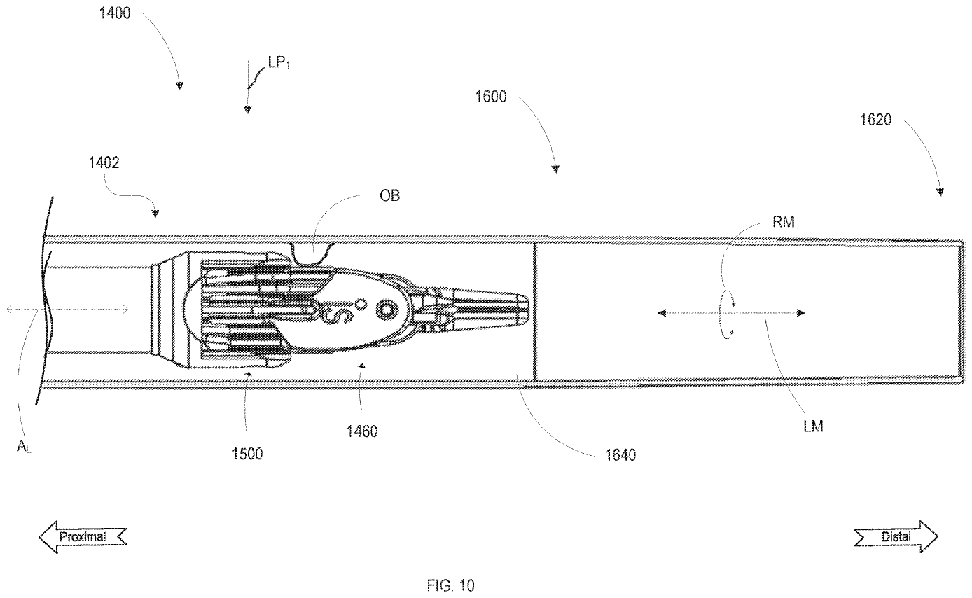

FIG. 10 is a cross-sectional side view of a portion of the cannula 1600 with a distal end portion 1402 of the instrument 1400 positioned therein and in contact with an object OB during at least a portion of a movement. The object OB can include a portion of the cannula 1600 (e.g., a wall of the central channel 1640 contacted by the end effector 1460) or debris, such as biologic tissue or a surgical material within the central channel 1640. In some embodiments, a portion of the movement of the distal end portion 1402 within the central channel 1640 can be affected by contact with the object OB. The contact with the object OB during a portion of the movement within the central channel 1640 can be sensed (e.g., measured) by the force sensor unit 1850 as a load affecting the instrument 1400. Thus, contact between the distal end portion 1402 and the object OB can affect the confirmation or reestablishment of the bias of the force sensor unit 1850. Utilizing the systems and methods described herein can facilitate the confirmation or reestablishment of the zero-offset of the force sensor unit 1850 even when a portion of the movement of the distal end portion 1402 is affected by contact with the object OB. Therefore, utilizing the systems and methods described herein can facilitate the accurate measurement of loads affecting the instrument 1400 and the provision of haptic feedback to the operator based on the measurements.

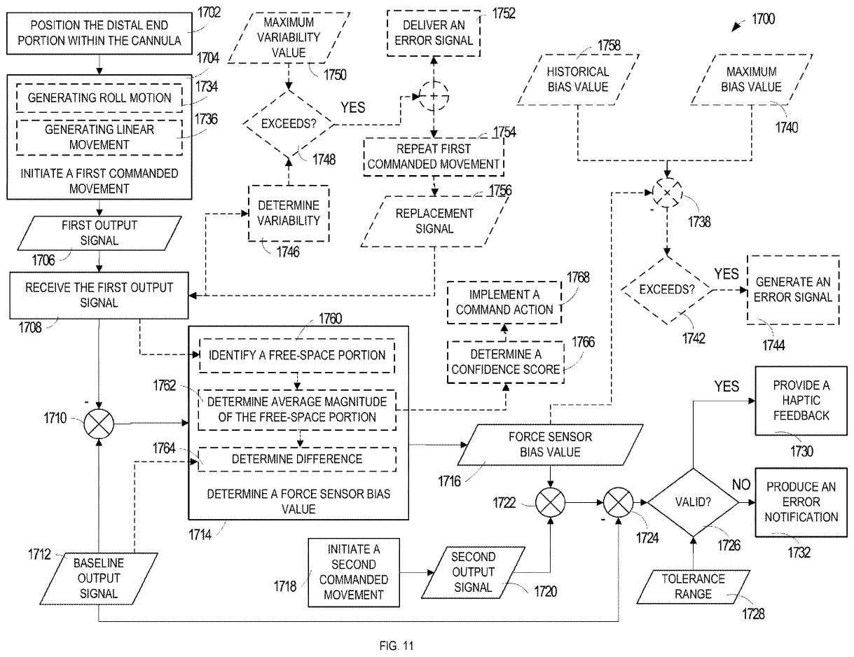

In some embodiments, the system 1000 includes the force sensor unit 1850 that is coupled to the instrument 1400 that is supported by the manipulator unit 1200. The input device 1116 is operably coupled to the instrument 1400 and the manipulator unit 1200 as previously described. The system 1000 includes a controller 1800 that is operably coupled to the manipulator unit 1200, the input device 1116, and the force sensor unit 1850. As further described below, the controller 1800 includes at least one processor 1802 and a haptic feedback module 1820. The controller 1800 is configured to perform a set of operations 1700, such as depicted in FIG. 11.

In some embodiments, the controller 1800 is configured to execute the set of operations 1700 upon receipt of a human command (e.g., a user input). For example, during a procedure, an operator of the system 1000 can input a recalibration command that triggers the implementation of the set of operations 1700, or any of the operations described herein. In additional embodiments, the controller 1800 is configured to detect the installation of the instrument 1400 on the instrument carriage 1330 of the manipulator unit 1200 and implement the set of operations 1700 (or any of the operations described herein) in response thereto. In still further embodiments, the controller 1800 is configured to implement the set of operations 1700 (or any of the operations described herein) based upon parameters of a signal received from the force sensor unit 1850 indicative of an anomaly with the force sensor unit 1850.

As depicted in FIG. 11 at 1702, in some embodiments, the set of operations 1700 includes positioning the distal end portion 1402 of the instrument 1400 within the cannula 1600, such as described with reference to FIGS. 9 and 10. With the distal end portion 1402 positioned within the cannula 1600 (e.g., within the central channel 1640), the controller 1800 initiates a first commanded movement at 1704. The first commanded movement of the distal end portion 1402 can include a roll motion RM about the shaft axis A_{L} (e.g., longitudinal axis) (FIG. 9), a linear movement LM along the shaft axis A_{L} (FIG. 9), a rotation of the end effector 1460 relative to the beam coordinate system BCS, a gripping via the end effector 1460, or any combination of these movements.

At 1708, a first output signal 1706 is received by the controller 1800 from the force sensor unit 1850. The first output signal 1706 is in response to the first commanded movement of the distal end portion 1402 of the instrument 1400 within the cannula 1600. The first output signal 1706 corresponds to the forces (e.g., loads) affecting the distal end portion 1402 within the cannula 1600 as perceived by the force sensor unit 1850. Under the nominal no-load condition within the central channel 1640, the initial zero-offset will result in a first output signal 1706 that indicates, with only negligible deviations, a load magnitude of “zero” in the absence of drift or other deviation of the bias. At 1710, the controller 1800 determines a difference between a portion of the first output signal 1706 and a baseline output signal 1712 for the force sensor unit 1850. The baseline output signal 1712 corresponds to an output signal of the force sensor unit 1850 as modified by the initial zero-offset. For example, under the nominal no-load condition within the central channel 1640, the baseline output signal 1712 has a load magnitude that is within a specified deviation from zero. For example, the baseline output signal 1712 can indicate a force magnitude of between 0.2 N and −0.2 N (e.g., 0.1 N to −0.1 N). The baseline output signal 1712 can be specific to the particular instrument 1400 coupled to the manipulator unit 1200.

As depicted at 1714, the controller 1800 determines a force sensor bias value 1716 based on the difference between the portion of the first output signal 1706 and the baseline output signal 1712. The force sensor bias value 1716, in combination with the initial zero-offset, can have a magnitude that, when applied to the output from the force sensor unit 1850 under a no-load condition, results in an output signal indicating, with only negligible deviations, a load magnitude of zero.

Referring still to FIG. 11, in some embodiments, the set of operations 1700 includes initiating a second commanded movement at 1718. In response to the second commanded movement, the controller 1800 receives a second output signal 1720 from the force sensor unit 1850. At 1722, the second output signal 1720 is modified by the force sensor bias value 1716. For example, the force sensor bias value 1716 is added to the second output signal 1720 generated by the force sensor unit 1850 in response to the second commanded movement. At 1724, the controller 1800 determines a deviation magnitude between the second output signal 1720 and the baseline output signal 1712. Based on the deviation magnitude, the controller 1800, at 1726, determines whether the force sensor bias value 1716 is valid. The force sensor bias value 1716 is valid on a condition that the deviation magnitude is within a predefined tolerance range 1728. In other words, the application of a valid force sensor bias value 1716 to the second output signal 1720 under a no-load condition, results in an output signal that has a magnitude deviation from zero that falls within the predefined tolerance range 1728. For example, the predefined tolerance range 1728 may require that the output of the force sensor unit 1850 be within 0.2 N (e.g., 0.1 N or less) of zero under a no-load condition. In some embodiments, the controller can simulate the second commanded movement and utilize previously recorded data to determines whether the force sensor bias value 1716 is valid.

On a condition that the force sensor bias value 1716 is valid, the set of operations 1700, at 1730, includes providing, via a haptic feedback module 1820 of the controller 1800, a haptic feedback to the user control unit 1100 (e.g., the input device 1116). The haptic feedback provided to the user control unit 1100 by the haptic feedback module 1820 is based on a load indication from the force sensor unit 1850 as modified by the force sensor bias value 1716. Said another way, the output from the force sensor unit 1850 is corrected by the force sensor bias value 1716 to provide an accurate indication of the loads affecting the instrument 1400 under a load condition. The corrected indication of the load is then utilized by the controller 1800 to provide accurate haptic feedback to the operator of the system 1000.

On a condition that the force sensor bias value 1716 is invalid, the set of operations 1700 includes producing an error signal (e.g., an error notification) at 1732. The force sensor bias value 1716 is invalid when the deviation magnitude between the second output signal 1720, as modified by the force sensor bias value 1716, and the baseline output signal 1712 is greater than the tolerance range. For example, in some embodiments, the force sensor bias value 1716 is invalid if the deviation magnitude is greater than 0.2 N. Said another way, the force sensor bias value 1716 is invalid if the magnitude of the force sensor bias value 1716 is insufficient to correct for any shift or drift of the force sensor unit 1850 from the initial zero-offset. In some embodiments, the error signal can include a visual indication, an audible indication, a haptic indication, or a combination thereof configured to alert an operator to the error condition. In some embodiments, a commanded action is implemented in response to the error condition. For example, in response to the error signal, the operator can remove the instrument 1400 from the manipulator unit 1200 and reinstall the instrument 1400. In some embodiments, in response to the error signal, the operator can withdraw the instrument 1400 from the cannula 1600 and clear an obstruction (e.g., object OB) from the cannula 1600. In some embodiments, the error signal can be indicative of a fault with the force sensor unit 1850 necessitating removing the instrument 1400 from service.

In some embodiments, on a condition in which the difference between the second output signal 1720 and the baseline output signal 1712 falls outside the tolerance range 1728, the controller 1800 generates an error signal to an operator of the system 1000 and implements a command action based, at least in part, on the error signal. In some embodiments, implementing the command action includes delivering an instruction to remove the instrument 1400 from the manipulator unit 1200 (e.g., the arm assembly 1300) and reinstall the instrument 1400. In additional embodiments, implementing the command action can include repeating the first commanded movement of the distal end portion 1402 within the cannula 1600 to generate a replacement first output signal. The controller 1800 can then determine a replacement force sensor bias value based on a difference between a portion of the replacement first output signal and the baseline output signal 1712. The haptic feedback is provided to the user control unit 1100 based on the load indication from the force sensor unit 1850 as modified by the replacement force sensor bias value. Further, in some embodiments, implementing the command action includes delivering an instruction to the operator of the system 1000 to remove the instrument 1400 from service due to a fault condition with the force sensor unit 1850.

Referring still to FIG. 11, in some embodiments, initiating the first commanded movement, at 1704, includes generating a roll motion RM (see e.g., FIGS. 9 and 10) of the distal end portion 1402 at 1734. The roll motion RM is rotational motion about the shaft axis A_{L} (e.g., the longitudinal axis) of the shaft 1410. The roll motion RM can be from a first roll limit, through a neutral orientation, to a second roll limit, and back to the neutral roll orientation. For example, the first roll limit can be achieved after up to 360 degrees (e.g., 350 degrees) of roll in a clockwise direction from a neutral roll orientation (e.g., a zero-degree position), while the second roll limit can be achieved after up to 360 degrees (e.g., 350 degrees) of roll in a counterclockwise direction from the neutral roll orientation. Accordingly, the roll motion RM can include up to 1080 degrees of rotation about the shaft axis A_{L} from the first roll limit to the second roll limit, and back to the neutral roll orientation. The controller 1800 is configured to maintain the distal end portion 1402 of the instrument 1400 within the cannula 1600 throughout the roll motion RM. For example, the controller 1800 is configured to maintain the distal end portion 1402 at the first longitudinal position LP₁ during the entirety of the roll motion RM.

In some embodiments, initiating the first commanded movement, at 1704, includes generating a linear movement LM (see e.g., FIGS. 9 and 10) of the distal end portion 1402 at 1736. The linear movement LM is translation movement parallel to the shaft axis A_{L} (e.g., the longitudinal axis) of the shaft 1410. The controller 1800 is configured to maintain the distal end portion 1402 of the instrument 1400 within the cannula 1600 throughout the linear movement LM. For example, the controller 1800 is configured to move the distal end portion 1402 from the first longitudinal position LP₁ to a second longitudinal position within the central channel 1640 of the cannula 1600.

As described herein with reference to FIGS. 5 and 6, the manipulator unit 1200 includes an instrument carriage 1330. The instrument carriage 1330 includes a set of drive discs 1320. Each individual drive disc 1320 is coupled to an individual motor 1340. The instrument 1400 includes a set of instrument discs 1474. Each individual instrument disc 1474 is configured to engage the corresponding drive disc 1320. The instrument discs 1474 are configured to receive motion from the drive discs 1320 to move the distal end portion 1402. Accordingly, the set of operations 1700 includes detecting the installation of the instrument 1400 on the instrument carriage 1330. Upon detecting the installation, the controller 1800 initiates an engagement process for the instrument 1400. The engagement process includes rotating at least one of the drive discs 1320 via the motors 1340 until the drive disc(s) 1320 engages the corresponding instrument disc 1474. The rotation of the drive disc(s) 1320 is continued until a stop condition is achieved for the drive disc(s) 1320.

The set of drive discs 1320 can include a roll-drive disc 1350. For example, as depicted in FIGS. 5 and 6, the system 1000 can include one roll-drive disc 1350, one roll-drive instrument disc 1476, four non-roll-drive discs, and four non-roll instrument discs. The roll-drive disc 1350 is configured to be operably coupled to the roll-drive instrument disc 1476 to generate the roll motion RM of the distal end portion 1402 about the shaft axis A_{L}. The non-roll-drive discs 1474 are each configured to be operably coupled to a corresponding drive disc 1320 such that rotation of the non-roll-drive discs produces movement of the end effector 1460 (e.g., pitch, yaw, or grip) about axes of a beam coordinate system BCS (see FIG. 6). In some embodiments, the engagement process includes maintaining the roll-drive disc 1350 at the first roll limit while at least one non-roll-drive disc of the set of drive discs 1320 is rotated to a neutral position (e.g., with the wrist 1500 at a pitch angle and a yaw angle of zero degrees). With the non-roll-drive disc at a neutral position, the first commanded movement is executed by generating the roll motion RM of the distal end portion 1402 from the first roll limit, through the roll range of motion, to the second roll limit. In some embodiments, executing the first commanded movement may include further rolling the distal end portion 1402 from the second roll limit to the neutral position.

In some embodiments, the instrument carriage 1330 is configured to maintain the distal end portion 1402 of the instrument 1400 within the cannula 1600 while moving the distal end portion 1402 in a proximal direction and in a distal direction. Thus, in some embodiments, the engagement process includes moving the distal end portion 1402 parallel to the shaft axis A_{L} from the first longitudinal position LP₁ to a second longitudinal position within the cannula 1600. In some embodiments, the distal end portion 1402 is then returned to the first longitudinal position LP₁. This longitudinal movement of the distal end portion 1402 can facilitate the measuring of forces exerted on the instrument shaft 1410 by a cannula seal (not shown). On a condition that the forces exerted by the cannula seal exceed a predetermined threshold, an error signal can be generated.

Referring again to FIG. 11, in some embodiments, the controller 1800 is configured to determine, at 1738, a difference between a magnitude of the force sensor bias value 1716 and a defined maximum force sensor bias value 1740. The maximum force sensor bias value 1740 corresponds to a maximal cumulative bias magnitude that can be applied to the output of the force sensor unit 1850 without affecting the accuracy of the representation of the forces affecting the instrument 1400. Based on the difference, the controller 1800, at 1742, determines whether the magnitude of the force sensor bias value 1716 exceeds the maximum force sensor bias value 1740. When the magnitude of the force sensor bias value 1716 exceeds the maximum force sensor bias value 1740, the controller 1800, at 1744, generates an error signal to an operator of the system 1000. The error signal can, for example, include instruction to operator to remove the instrument 1400 from the manipulator unit 1200 and reinstalled instrument 1400. Alternatively, the error signal can, direct the operator to remove the instrument 1400 from service. As a further alternative, in response to the error signal, the operator can at least partially disable the haptic feedback and utilize the instrument 1400 to perform an operation.

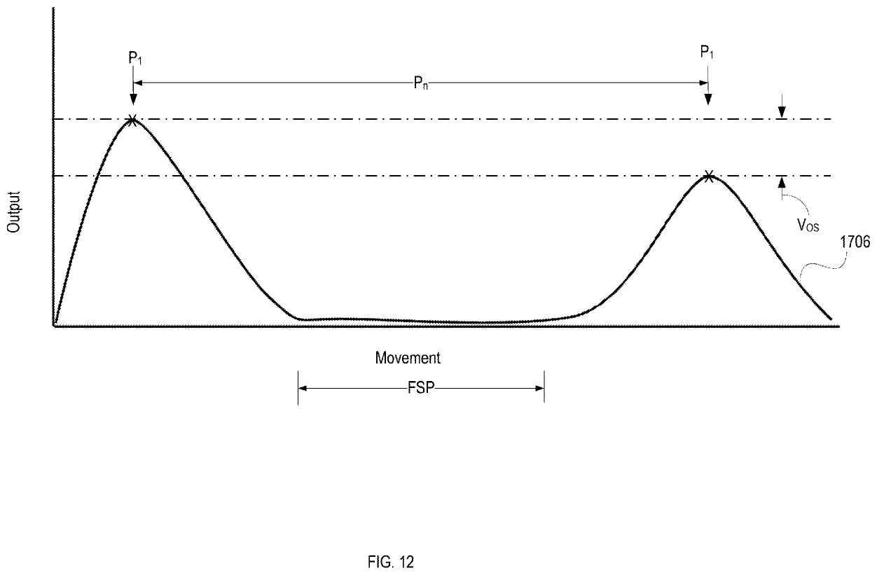

Referring to FIG. 11 and also to FIG. 12, in some embodiments, initiating the first commanded movement, at 1704, includes establishing the distal end portion 1402 in a first pose P₁ (e.g., a first kinematic pose). For example, establishing the distal end portion 1402 in the first pose P₁ can include positioning the distal end portion 1402 at the first longitudinal position LP₁ with a specified degree (e.g., 30, 40, 50, etc. degrees) of roll from the neutral roll orientation. Establishing the first pose P₁ can include positioning the end effector 1460 at a first pitch, yaw, and grip setting. The first commanded movement also includes transitioning the distal end portion 1402 away from the first pose P₁ to any additional pose Pₙ and returning the distal end portion 1402 to the first pose P₁ as depicted in FIG. 12. At 1746, the controller 1800 determines a variability Vos of the first output signal 1706 between each instance of the distal end portion 1402 in the first pose P₁. At 1748, the controller 1800 determines whether the variability V_{OS} exceeds a maximum variability value 1750 (e.g., a maximal variability threshold). On a condition in which the variability V_{OS} exceeds the maximum variability value 1750, the controller 1800, at 1752, delivers an error signal to an operator of the system 1000. In some embodiments, on a condition in which the variability V_{OS} exceeds the maximum variability value 1750, the first commanded movement is repeated, at 1754, to generate a replacement first output signal 1756 and the force sensor bias value 1716 is determined (as described herein) based, at least in part, on the replacement first output signal 1756.

As depicted in FIG. 11, in some embodiments, the controller 1800 is configured to determine a variance of the magnitude of the force sensor bias value 1716 relative to a historical force sensor bias value 1758. The historical force sensor bias value 1758 can include force sensor bias values recorded during previous installations of the particular instrument 1400. A force sensor bias value 1716 for the present installation of the instrument 1400 that deviates significantly from the force sensor bias values that have been previously utilized for the particular instrument 1400 can be indicative of a failed or failing component of the instrument 1400, such as the force sensor unit 1850. Accordingly, on a condition in which the variance exceeds a variance threshold, the controller 1800 delivers an error signal to operator of the system 1000. In some embodiments, the error signal indicates a fault with the force sensor unit 1850.

In some embodiments, the historical force sensor bias value 1758 can be a valid force sensor bias value 1716 recorded during the installation of the particular instrument 1400 at the initiation of a current surgical procedure. Said another way, as the instrument 1400 can be processed (e.g., sterilized) prior to the initiation of each surgical procedure the historical force sensor bias value 1758 can be the first valid force sensor bias value 1716 recorded following the immediately preceding processing of the instrument 1400. In the event that the instrument 1400 has not been exposed to the relatively high heat of reprocessing during the current surgical procedure, in some embodiments, the valid force sensor bias value 1716 recorded during the installation of the particular instrument 1400 at the initiation of the current surgical procedure can be used to correct the output from the force sensor unit 1850. For example, during a single surgical procedure, a particular instrument 1400 can be installed and a valid force sensor bias value 1716 can be recorded during a first portion of the procedure. This valid force sensor bias value 1716 can be considered to be the historical force sensor bias value 1758. The instrument 1400 can then be removed from the manipulator unit 1200 as other portions of the procedure are performed. During a later portion of the procedure, the instrument 1400 can be re-installed and an invalid force sensor bias value (e.g., modified output signal has a magnitude deviation from zero that falls outside the tolerance range) can be computed. In the event of such an occurrence, the controller 1800 can generate a notification to the operator of the system 1000 and apply the historical force sensor bias value 1758 to the output from the force sensor unit 1850. Accordingly, the output from the force sensor unit 1850 is corrected by the historical force sensor bias value 1758 to provide an accurate indication of the loads affecting the instrument 1400 under a load condition. The corrected indication of the load is then utilized by the controller 1800 to provide accurate haptic feedback to the operator of the system 1000.

As depicted in FIG. 6, the instrument 1400 includes a beam coordinate system BCS. The beam coordinate system BCS includes a first axis, a second axis, and a third axis that are orthogonal to one another. As such, in some embodiments, the force sensor bias value 1716 is a first force sensor bias value that is parallel to the first axis. Accordingly, the set of operations 1700 includes resolving the first output signal 1706 in the beam coordinate system BCS to determine a first axis component, a second axis component, and a third axis component of the first output signal 1706. The controller 1800 is then configured to determine a second force sensor bias value that is parallel to the second axis based on a difference between a portion of the second axis component and a baseline second axis component. The controller 1800 can also be configured to determine a third force sensor bias value parallel to the third axis component and a baseline third axis component. In other words, the set of operations 1700 can include determining a force sensor bias value 1716 in each of the orthogonal axes of the beam coordinate system BCS. Each of the first force sensor bias value, the second force sensor bias value, and the third force sensor bias value can have a magnitude that differs from at least one other force sensor bias value. For example, the first force sensor bias value can have a first magnitude, the second force sensor bias value can have a second magnitude, and the third force sensor bias value can have a third magnitude, with each magnitude being different.

As described previously, the controller 1800 determines force sensor bias value 1716 based on a difference between a portion of the first output signal 1706 and the baseline output signal 1712. In some embodiments, the portion of the first output signal 1706 is associated with the instrument 1400 (e.g., the distal end portion 1402) being in a specific sampling pose. In some embodiments, the specific sampling pose includes a specified roll orientation of the distal end portion 1402. The specified roll orientation can correspond to a defined zero orientation (e.g., the neutral roll orientation). For example, in some embodiments, following the installation of the instrument 1400, each of the non-roll-drive discs is rotated to a neutral position to place the wrist assembly 1500 and the end effector 1460 in a neutral position and into alignment with the shaft axis A_{L}. Said another way, the non-roll-drive discs are rotated such that the pitch angle is zero, the jaws are closed, and the yaw angle is zero. The distal end portion 1402 is then rolled through at least a portion of the roll range of motion to achieve the defined zero orientation. The defined zero orientation can include an arc of degrees (e.g., 5 degrees or less) extending on either side of a zero-degree point. The arc of degrees can be five degrees or less (e.g., two degrees) on either side of the zero-degree point. The arc of degrees can be utilized for signal-noise accommodation. In some embodiments, the portion of the first output signal 1706 can be received only when the distal end portion 1402 is oriented at the zero-degree point. In other embodiments, the portion of the first output signal 1706 can be associated with a specified time interval following installation of the instrument 1400 or other suitable initiation event.

As depicted in FIG. 11 at 1760, in some embodiments, determining the force sensor bias value 1716 includes identifying a free-space portion FSP of the first output signal 1706. In some embodiments, the free-space portion FSP corresponds to a portion of the first output signal 1706 that has a fit line with the slope that is less than a defined slope threshold over a specified minimum time interval as depicted in FIG. 12. Said another way, the free-space portion FSP can correspond to the flattest and/or most horizontal region of the first output signal 1706 when depicted graphically as in FIG. 12. The free-space portion FSP can be identified (e.g., selected or defined) via any suitable means, such as graphically, algorithmically, and/or manually.

The free-space portion FSP of the first output signal 1706 corresponds to a free-space condition of the distal end portion 1402 of the instrument 1400. As depicted in FIG. 9, the free-space condition of the distal end portion 1402 indicated by the free-space portion FSP is a condition of the instrument 1400 in which the first commanded movement of the instrument 1400 (e.g., the distal end portion 1402) is not affected by contact with another object OB (see e.g., FIG. 10). The free-space portion FSP can, therefore, correspond to a no-load condition. Accordingly, at 1762, the controller 1800 can determine an average magnitude of the free-space portion FSP. At 1764, the controller 1800 determines a difference between the average magnitude of the free-space portion FSP of the first output signal 1706 and the baseline output signal 1712 for the force sensor 1850. In some embodiments, the force sensor bias value 1716 corresponds to the difference between the average magnitude of the free-space portion FSP of the first output signal 1706 and the baseline output signal 1712.

Referring still to FIG. 11, in some embodiments, the controller 1800, at 1766, is configured to determine a confidence score for the free-space portion FSP of the first output signal 1706. The confidence score is indicative of a correlation between the free-space portion FSP and a condition of the instrument 1400 in which the first commanded movement is not affected by contact with another object OB. For example, a free-space portion FSP that has minimal deviation from a slope of zero over a time interval that exceeds the minimum time interval can be assigned a confidence score approaching one. A confidence score approaching one, indicates a relatively high likelihood that the first commanded movement is not affected by contact with another object OB. However, a free-space portion FSP that has greater deviations from a slope of zero can be assigned a lower confidence score indicating a lower likelihood of a free-space condition.

In some embodiments, the controller 1800, at 1768, implements a command action based, at least in part, on the confidence score. For example, on a condition in which the confidence score is less than a confidence score threshold, the command action can include repeating the first commanded movement of the distal end portion 1402 within the cannula 1600 to generate a replacement first output signal. The controller 1800 can then identify a replacement free-space portion FSP of the replacement first output signal and determine the force sensor bias value based, at least in part, on the replacement free-space portion FSP. In additional embodiments, on a condition in which the confidence score is less than the confidence score threshold, implementing the command action can include delivering an error signal to the user control unit 1100. In further embodiments, on a condition which the confidence score is less than the confidence score threshold, the command action can include generating a maintenance alert that is indicative of a failed or failing force sensor unit 1850.

In some embodiments, implementing the command action can include applying a gain value to the haptic feedback that is provided to the input device 1116. The gain value can be determined based, at least in part, on the confidence score. For example, on a condition in which the confidence score approaches one, the nominal (e.g., designed) haptic feedback for the applied forces affecting the instrument 1400 can be delivered to the input device 1116. However, on a condition in which the confidence score is less than one, the gain value can be applied to reduce the haptic feedback delivered to the input device 1116 for the same applied forces.

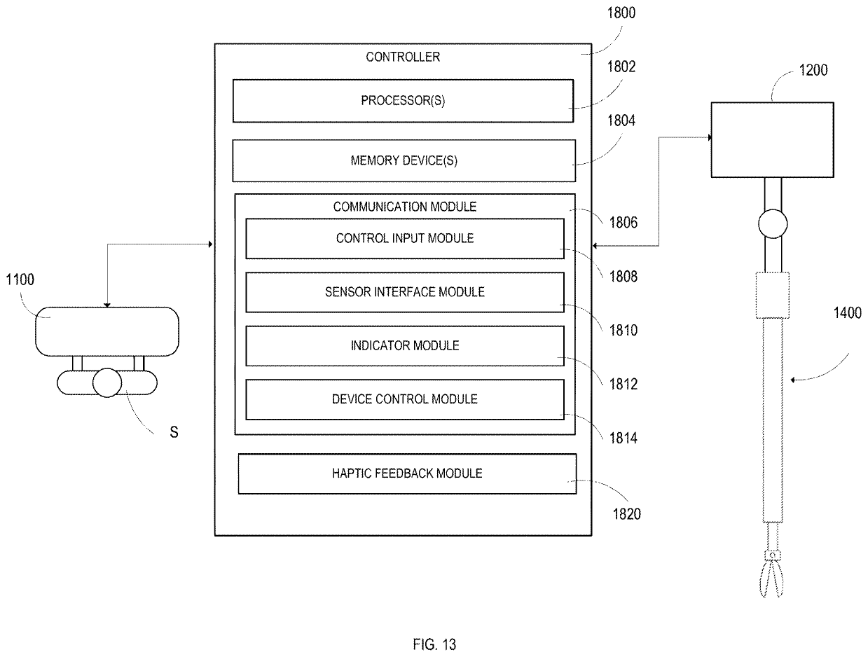

As shown particularly in FIG. 13, a schematic diagram of one embodiment of suitable components that may be included within the controller 1800 is illustrated. In some embodiments, the controller 1800 is positioned within a component of the surgical system 1000, such as the user control unit 1100 and/or the optional auxiliary equipment unit 1150. However, the controller 1800 may also include distributed computing systems wherein at least one aspect of the controller 1800 is at a location which differs from the remaining components of the surgical system 1000 for example, at least a portion of the controller 1800 may be an online controller.

As depicted, the controller 1800 includes one or more processor(s) 1802 and associated memory device(s) 1804 configured to perform a variety of computer implemented functions (e.g., performing the methods, steps, calculations and the like and storing relevant data as disclosed herein). Additionally, in some embodiments, the controller 1800 includes a communication module 1806 to facilitate communications between the controller 1800 and the various components of the surgical system 1000.

As used herein, the term “processor” refers not only to integrated circuits referred to in the art as being included in a computer, but also refers to a controller, a microcontroller, a microcomputer, a programmable logic controller (PLC), an application specific integrated circuit, and other programmable circuits. Additionally, the memory device(s) 1804 may generally comprise memory element(s) including, but not limited to, computer readable medium (e.g., random access memory (RAM)), computer readable nonvolatile medium (e.g., a flash memory), a floppy disc, a compact disc read only memory (CD ROM), a magneto optical disc (MOD), a digital versatile disc (DVD) and/or other suitable memory elements. Such memory device(s) 1804 may generally be configured to store suitable computer readable instructions that, when implemented by the processor(s) 1802, configure the controller 1800 to perform various functions.

In some embodiments, the controller 1800 includes a haptic feedback module 1820. The haptic feedback module 1820 may be configured to deliver a haptic feedback to the operator based on inputs received from a force sensor unit 1850 of the instrument 1400. In some embodiments, haptic feedback module 1820 may be an independent module of the controller 1800. However, in some embodiments the haptic feedback module 1820 may be included within the memory device(s) 1804.

The communication module 1806 may include a control input module 1808 configured to receive control inputs from the operator/surgeon S, such as via the input device 1116 of the user control unit 1100. The communication module may also include an indicator module 1812 configured to generate various indications in order to alert the operator.

The communication module 1806 may also include a sensor interface 1810 (e.g., one or more analog to digital converters) to permit signals transmitted from one or more sensors (e.g., strain sensors of the force sensor unit 1850) to be converted into signals that can be understood and processed by the processors 1802. The sensors may be communicatively coupled to the communication module 1806 using any suitable means. For example the sensors may be coupled to the communication module 1806 via a wired connection and/or via a wireless connection, such as by using any suitable wireless communications protocol known in the art. Additionally, in some embodiments, the communication module 1806 includes a device control module 1814 configured to modify an operating state of the instrument 1400 (and/or any of the instruments described herein. Accordingly, the communication module is communicatively coupled to the manipulator unit 1200 and/or the instrument 1400. For example, the communications module 1806 may communicate to the manipulator unit 1200 and/or the instrument 1400 an excitation voltage for the strain sensor(s), a handshake and/or excitation voltage for a positional sensor (e.g., for detecting the position of the designated portion relative to the cannula), cautery controls, positional setpoints, and/or an end effector operational setpoint (e.g., gripping, cutting, and/or other similar operation performed by the end effector).

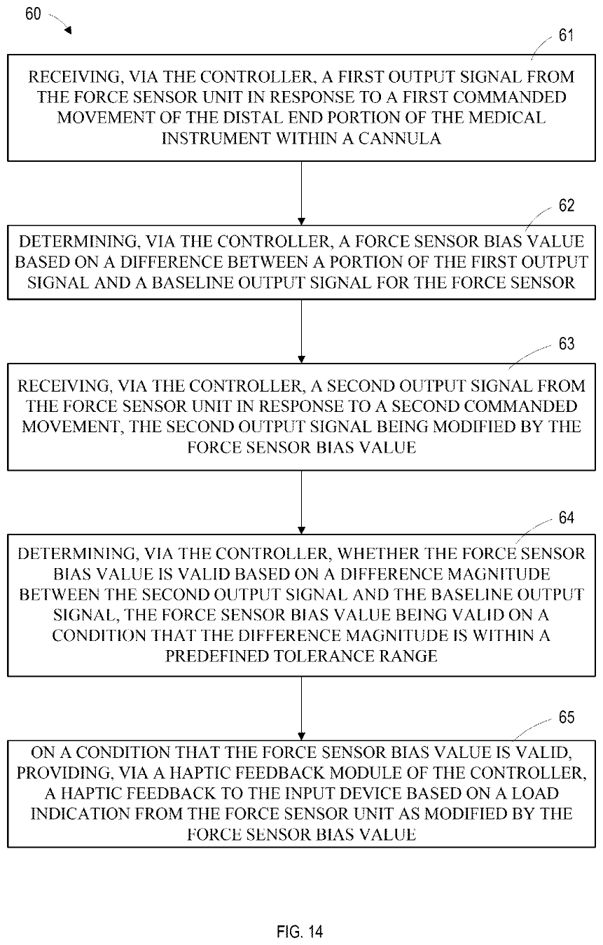

FIG. 14 is a flow chart of a method 60 of control for a surgical system according to an embodiment. The method 60 may, in an embodiment, be performed via a teleoperated system, such as system 1000 as described with reference to FIGS. 1-13. However, it should be appreciated that in various embodiments, aspects of the method 60 may be accomplished via additional embodiments of the system 1000 or components thereof as described herein. Accordingly, the method 60 may be implemented on any suitable device as described herein. Thus, the method 60 is described below with reference to instrument 1400 and the controller 1800 of the system 1000 as previously described, but it should be understood that the method 60 can be employed using any of the medical devices/instruments and controllers described herein.

As depicted at 61, the method 60 includes receiving, via the controller, a first output signal from the force sensor unit in response to a first commanded movement of the distal end portion of the medical instrument within a cannula. As depicted at 62, the method 60 includes determining, via the controller, a force sensor bias value based on a difference between a portion of the first output signal and a baseline output signal for the force sensor unit. As depicted at 63, the method 60 includes receiving, via the controller, a second output signal from the force sensor unit in response to a second commanded movement. The second output signal is modified by the force sensor bias value. As depicted at 64, the method 60 includes determining, via the controller, whether the force sensor bias value is valid based on a deviation magnitude between the second output signal and the baseline output signal. The force sensor bias value is valid on a condition that the deviation magnitude is within a predefined tolerance range. On a condition that the force sensor bias value is valid, the method 60, as depicted at 65, includes providing, via a haptic feedback module of the controller, a haptic feedback to the user control unit based on a load indication from the force sensor unit as modified by the force sensor bias value.

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a surgical assembly that performs minimally invasive surgical procedures, and which can include a manipulator unit, a series of kinematic linkages, a set of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body or the like.

For example, any of the components of a surgical instrument as described herein can be constructed from any material, such as medical grade stainless steel, nickel alloys, titanium alloys or the like. Further, any of the links, tool members, beams, shafts, cables, or other components described herein can be constructed from multiple pieces that are later joined together. For example, in some embodiments, a link can be constructed by joining together separately constructed components. In other embodiments however, any of the links, tool members, beams, shafts, cables, or components described herein can be monolithically constructed.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

1-54. (canceled)

55. A method of control for a surgical system, the surgical system including a manipulator unit, a controller, a user control unit, and a medical instrument supported by the manipulator unit and operably coupled to be controlled by the user control unit via the controller, the medical instrument including a force sensor unit, the method comprising:
receiving, via the controller, a first output signal from the force sensor unit in response to a first commanded movement of a distal end portion of the medical instrument within a cannula;
determining, via the controller, a force sensor bias value based on a difference between a portion of the first output signal and a baseline output signal for the force sensor unit;
receiving, via the controller, a second output signal from the force sensor unit in response to a second commanded movement, the second output signal being modified by the force sensor bias value;
determining, via the controller, whether the force sensor bias value is valid based on a deviation magnitude between the second output signal and the baseline output signal, the force sensor bias value being valid on a condition that the deviation magnitude is within a predefined tolerance range; and
on a condition that the force sensor bias value is valid, providing, via a haptic feedback module of the controller, a haptic feedback to the user control unit based on a load indication from the force sensor unit as modified by the force sensor bias value.

56. The method of claim 55, wherein:
the first commanded movement includes a roll motion of the distal end portion about a longitudinal shaft axis from a first roll limit, through a neutral roll orientation, to a second roll limit, and back to the neutral roll orientation; and
the controller maintains the distal end portion of the medical instrument within the cannula throughout the roll motion.

57. The method of claim 55, wherein:
the manipulator unit includes a plurality of motors and a plurality of drive discs;
each individual drive disc of the plurality of drive discs is coupled to a corresponding individual motor of the plurality of motors;
the medical instrument includes a plurality of instrument discs configured to receive motion from the plurality of drive discs to move the distal end portion;
each individual instrument disc of the plurality of instrument discs is configured to engage a corresponding individual drive disc of the plurality of drive discs; and
the method includes:
detecting, via the controller, an installation of the medical instrument on the manipulator unit,
initiating, via the controller, an engagement process for the medical instrument in response to detecting the installation, and
rotating, via the controller, at least one of the plurality of drive discs via the plurality of motors until the drive disc engages the corresponding instrument disc and a stop condition is achieved for the drive disc.

58. The method of claim 57, wherein:
the plurality of drive discs includes a roll-drive disc configured to generate a roll motion of the distal end portion of the medical instrument about a longitudinal shaft axis; and
the method includes:
maintaining the roll-drive disc at a first roll limit,
rotating at least one non-roll-drive disc of the plurality of drive discs to a neutral position, and
executing the first commanded movement by generating the roll motion of the distal end portion through a roll range of motion to a second roll limit.

59. The method of claim 55, wherein:
the method includes:
determining, via the controller, a difference between a magnitude of the force sensor bias value and a defined maximum force sensor bias value, and
on a condition in which the magnitude of the force sensor bias value exceeds the maximum force sensor bias value, providing, via the controller, an error signal to an operator of the surgical system.

60. The method of claim 55, wherein:
the first commanded movement includes establishing the distal end portion of the medical instrument in a first pose, transitioning the distal end portion away from the first pose, and returning the distal end portion to the first pose; and
the method includes:
determining, via the controller, a variability of the first output signal between each instance of the distal end portion in the first pose, and
on a condition in which the variability exceeds a maximum variability value, providing, via the controller, an error signal to an operator of the surgical system.

61. The method of claim 60, wherein:
the method includes:
on a condition in which the variability exceeds the maximum variability value, repeating the first commanded movement of the distal end portion of the medical instrument within the cannula to generate a replacement first output signal, and
determining, via the controller, the force sensor bias value based at least in part on the replacement first output signal.

62. The method of claim 55, wherein:
the medical instrument includes a beam coordinate system having a first axis, a second axis, and a third axis that are orthogonal to one another;
the force sensor bias value is a first force sensor bias value that is parallel to the first axis; and
the method includes:
resolving, via the controller, the first output signal in the beam coordinate system to determine a first axis component, a second axis component, and a third axis component of the first output signal,
determining, via the controller, a second force sensor bias value parallel to the second axis based on a difference between a portion of the second axis component and a baseline second axis component, and
determining, via the controller, a third force sensor bias value parallel to the third axis based on a difference between a portion of the third axis component and a baseline third axis component.

63. The method of claim 55, wherein:
the portion of the first output signal is associated with the medical instrument being in a specified sampling pose.

64. The method of claim 63, wherein:
the specified sampling pose includes a roll orientation of the distal end portion of the medical instrument that corresponds to a defined zero orientation.

65. The method of claim 55, wherein:
determining the force sensor bias value includes identifying a free-space portion of the first output signal that corresponds to a free-space condition of the distal end portion of the medical instrument; and
the force sensor bias value corresponds to the difference between an average magnitude of the free-space portion of the first output signal and the baseline output signal for the force sensor unit.

66. The method of claim 65, wherein:
the method includes:
determining, via the controller, a confidence score for the free-space portion, and
implementing, via the controller, a command action based at least in part on the confidence score; and
the confidence score is indicative of a correlation between the free-space portion and a condition of the medical instrument in which the first commanded movement of the medical instrument is not affected by contact with another object.

67. The method of claim 66, wherein:
on a condition in which the confidence score is less than a confidence score threshold, implementing the command action includes:
repeating the first commanded movement of the distal end portion of the medical instrument within the cannula to generate a replacement first output signal,
identifying a replacement free-space portion of the first output signal, and
determining the force sensor bias value based at least in part on the replacement free-space portion.

68. The method of claim 55, the method includes:
on a condition in which the difference between the second output signal and the baseline output signal falls outside the tolerance range, providing, via the controller, an error signal to an operator of the surgical system; and
implementing a command action based at least in part on the error signal, implementing the command action includes:
repeating the first commanded movement of the distal end portion of the medical instrument within the cannula to generate a replacement first output signal,
determining, via the controller, a replacement force sensor bias value based on a difference between a portion of the replacement first output signal and the baseline output signal for the force sensor unit, and
providing, via the controller, the haptic feedback to the user control unit based on the load indication from the force sensor unit as modified by the replacement force sensor bias value.

69. A surgical system, comprising:
a medical instrument including a distal end portion, the medical instrument being supported by a manipulator unit;
a force sensor unit coupled to the medical instrument;
user control unit operably coupled to the medical instrument and the manipulator unit; and
a controller operably coupled to the manipulator unit, the user control unit, and the force sensor unit, the controller comprising at least one processor and a haptic feedback module, controller being configured to perform a plurality of operations, the plurality of operations comprising:
receiving a first output signal from the force sensor unit in response to a first commanded movement of the distal end portion of the medical instrument within a cannula,
determining a force sensor bias value based on a difference between a portion of the first output signal and a baseline output signal for the force sensor unit,
receiving a second output signal from the force sensor unit in response to a second commanded movement, the second output signal being modified by the force sensor bias value,
determining whether the force sensor bias value is valid based on a deviation magnitude between the second output signal and the baseline output signal, the force sensor bias value being valid on a condition that the deviation magnitude is within a predefined tolerance range, and
on a condition that the force sensor bias value is valid, providing, via the haptic feedback module, a haptic feedback to the user control unit based on a load indication from the force sensor unit as modified by the force sensor bias value.

70. The system of claim 69, wherein:
the first commanded movement includes a roll motion of the distal end portion about a longitudinal shaft axis from a first roll limit, through a neutral roll orientation, to a second roll limit, and back to the neutral roll orientation; and
the controller is configured to maintain the distal end portion of the medical instrument within the cannula throughout the roll motion.

71. The system of claim 69, wherein:
the first commanded movement includes a linear movement of the distal end portion parallel to a longitudinal shaft axis; and
the controller is configured to maintain the distal end portion of the medical instrument within the cannula throughout the linear movement.

72. The system of claim 69, wherein:
the manipulator unit includes a plurality of motors and a plurality of drive discs;
each individual drive disc of the plurality of drive discs is coupled to a corresponding individual motor of the plurality of motors;
the medical instrument includes a plurality of instrument discs configured to receive motion from the plurality of drive discs to move the distal end portion;
each individual instrument disc of the plurality of instrument discs is configured to engage a corresponding individual drive disc of the plurality of drive discs; and
the plurality of operations includes:
detecting an installation of the medical instrument on the manipulator unit,
initiating an engagement process for the medical instrument in response to detecting the installation, and
rotating at least one of the plurality of drive discs via the plurality of motors until the drive disc engages the corresponding instrument disc and a stop condition is achieved for the drive disc.

73. The system of claim 72, wherein:
the plurality of drive discs includes a roll-drive disc configured to generate a roll motion of the distal end portion of the medical instrument about a longitudinal shaft axis; and
the plurality of operations includes:
maintaining the roll-drive disc at a first roll limit,
rotating at least one non-roll-drive disc of the plurality of drive discs to a neutral position, and
executing the first commanded movement by generating the roll motion of the distal end portion through a roll range of motion to a second roll limit.

74. The system of claim 69, wherein:
the plurality of operations includes:
determining a difference between a magnitude of the force sensor bias value and a defined maximum force sensor bias value, and
on a condition in which the magnitude of the force sensor bias value exceeds the maximum force sensor bias value, providing an error signal to an operator of the surgical system.
